(19) United States
(12) Patent Application Publication
(10) Pub. No.: US 2014/0031490 A1
(43) Pub. Date: Jan. 30, 2014

(54) GLYCEROL BASED UNSATURATED POLYESTER RESINS AND RAW MATERIALS THEREFOR

(71) Applicants: Kim Pickering, Hamilton (NZ); Carmen Viljoen, Pretoria (ZA); Casparus Johannes Reinhard Verbeek, Hamilton (NZ); Christopher Brian King, Manukau (NZ)

(72) Inventors: Kim Pickering, Hamilton (NZ); Carmen Viljoen, Pretoria (ZA); Casparus Johannes Reinhard Verbeek, Hamilton (NZ); Christopher Brian King, Manukau (NZ)

(73) Assignees: WAIKATOLINK LIMITED, Hamilton (NZ); NUPLEX RESINS B.V., Bergen Op Zoom (NL)

(21) Appl. No.: 14/110,461

(22) PCT Filed: Apr. 4, 2012
(86) PCT No.: PCT/EP12/56188

§ 371 Date: Oct. 8, 2013

(30) Foreign Application Priority Data
Apr. 8, 2011 (EP) 11161644.7

(51) Int. Cl. C08G 63/85; C07C 67/08

(52) U.S. Cl. CPC C08G 63/85; C07C 67/08
USPC 524/600; 528/283; 525/437; 560/204

(57) ABSTRACT

The invention relates to a mixture of glycerol, mono-, di- and triacetylglycerolester in which the amount of tri-ester is less than 15 mol %, the amount of glycerol is less than 25 mol %, the amount of monoester is about 20 mol % or more, more preferred about 30 mol % or more and the amount of diester is about 20 mol % or more preferred about 40 mol % or more. The invention further relates to methods to prepare such glycerolacetylester mixtures, and to the use thereof in the preparation of unsaturated polyesters. Polyesters comprising said glycerolacetylester mixtures are made from a higher amount of raw materials than obtainable from renewable resources.

### BACKGROUND OF THE INVENTION

1. Field of the Invention

The invention relates to glycerol based unsaturated polyester resins and raw materials therefore.

2. Description of the Related Art

Unsaturated polyester resins are commonly used in constructive parts in for example building, automotive and shipbuilding industries.

One of the concerns in industry is the reliance on oil based resources. The environment would be aided if use could be made of raw materials of natural resources. Several studies exist on this subject. For example de Meireles Brioude et al. in ‘Synthesis and Characterization of Aliphatic Polyesters from Glycerol, by-Product of Biodiesel Production, and Adipic Acid’. Materials Research, (2007) 10, 335-339 describe the use of glycerol, a waste product from the production of biodiesel. Another example by Miyagawa, H. et al. in ‘Development of biobased unsaturated polyester containing functionalised linseed oil’. Ind. Eng. Chem. Res., (2006) 45, 1014-1018 describes the use of functionalized linseed oil as an addition material to traditional unsaturated polyester resins. However, the use of functionalized linseed oil causes a decrease in modulus.

There is an ongoing need for unsaturated polyester resins that use bioderivable raw materials.

### BRIEF SUMMARY OF THE INVENTION

The present invention relates to unsaturated polyester resins comprising a substantial amount of glycerol. Glycerol is a by-product from the manufacture of biodiesel, and is nowadays a cheap bioderived raw material. However, because glycerol is tri-functional, its use in polymer systems like unsaturated polyesters for making thermoset products through radical polymerization with high strength and modulus, has been very limited.

The present invention therefore furthermore relates to the use of glycerol mono- and diacetate (hereinafter also denoted as mono/di/tri acetylglycerol esters) in the manufacture of unsaturated polyester resins and other resins.

The present invention furthermore relates to the process of making mono and diacetyl esters of glycerol from acetic acid and glycerol with a relatively low molar ratio while using an organotin catalyst.

The present invention furthermore relates to the process of making an unsaturated polyester from an α,β-unsaturated carboxylic acid and at least a acetylglycerol esters, wherein mono- and diacetyl esters of glycerol are made from glycerol and acetic acid while using an organotin catalyst, and wherein in making the unsaturated polyester the same organotin catalyst is used.

### DETAILED DESCRIPTION OF THE INVENTION

In one embodiment of the invention, the invention relates to a process for making acetylesters of glycerol while using a stannous catalyst, the acetylester mixture being a mixture of non-reacted, mono-, di-, and/or triacetylglycerolester. This process for making a mixture of acetylesters of glycerol is performed with such an acetic acid/glycerol ratio that the product can be directly used in the unsaturated polyester manufacture. This is for example possible with ratio of acetic acid to glycerol of about 1.5 to 1 or higher, preferably 1.7 to 1 or higher. Generally, it is preferred to have this ration 2.5 to 1 or lower, preferably 2.2 to 1 or lower.

The use of a stannous catalyst allows a process with relatively high selectivity for mono- and diacetylesters. In particular for the present invention, that uses mono- and diacetylesters in unsaturated polyester synthesis, this selectivity is highly valuable. This is an advantage because triacetylester acts as a plasticizer, and glycerol acts as a branching agent, both of which generally are only allowable in a relatively low amount.

In another embodiment of the invention, the invention relates to a process for making glycerol, mono-, di- and triacetylglycerolester in which the amount of tri-ester produced relative to the resulting glycerol, mono- and di-ester is less than 15 mol %, preferably less than 10 mol %.

In another embodiment of the invention, the invention relates to a process for making glycerol, mono-, di- and triacetylglycerolester in which the amount of glycerol produced relative to the resulting mono-, di- and tri-ester is less than 25 mol %, preferably less than 15 mol % and more preferably less than 10 mol %.

In a preferred embodiment, the invention relates to a process for making glycerol, mono-, di- and triacetylglycerolester in which the amount of monoester produced relative to the resulting glycerol, di- and tri-ester is about 20 mol % or more, preferably about 25 mol % or more, and even more preferred about 30 mol % or more. The amount of mono-ester will generally be about 50 wt % or less, and may be about 40 wt % or less.

In a further preferred embodiment, the invention relates to a process for making glycerol, mono-, di- and triacetylglycerolester in which the amount of diester produced relative to the resulting glycerol, mono- and tri-ester is about 20 mol % or more, preferably about 30 mol % or more, and even more preferred about 40 mol % or more. The amount of di-ester will generally be about 60 wt % or less, and may be about 50 wt % or less.

In a further embodiment, the invention relates to a process for making glycerol, mono-, di- and triacetylglycerolester wherein the amount of triester produced produced relative to the resulting glycerol, mono- and di-ester is between 1.0 mol % and about 15 mol %, and wherein the amount of glycerol produced produced relative to the resulting mono-, di- and tri-ester is between 5.0 mol % and about 25 mol %,

The monoacetylester and diacetylester of glycerol can exist in two isomers (e.g. 1-monoacetylester and 2-monoacetylester, and 1,2-diacetylester and 1,3-diacetylester). For the present invention, mono- and diacetylester will be used.

The acetylester mixture of glycerol, with a free glycerol content of less than 15 mol %, monoester in an amount between 25-50 mol %, diester between 30-50 mol % and trimester in an amount less than 15 mol % is very useful in the preparation of polyesters, and in particular of unsaturated polyesters that can be polymerized through radical polymerization to form articles.

Glycerol as such is used in the preparation of polyesters, like trimethylolpropane, as a branching agent. However, such branching agent generally is used in an amount of less than 3 wt % relative to the polyols. Higher amounts may lead to gelling of the polyesters during synthesis.

Higher amounts of trifunctional alcohols can be used, if combined with monofunctional acids or other chain stoppers. Well known monofunctional acids are fatty acids, used in the preparation of alkyd resins for coatings. In unsaturated polyesters, such fatty acids lead to lowering of the modulus, and to increased flexibility. Hence, the use of fatty acids (and with it, substantial amounts of trifunctional alcohols) is not preferred. Generally—if used at all—, in unsaturated polyester synthesis, benzoic acid is used as monofunctional acid. Benzoic acid is made from benzene or toluene, being oil based raw materials.

The present invention allows substantial amounts of glycerol to be used in unsaturated polyesters, without one of the downsides of (i) much reduced tensile modulus, or (ii) the necessary use of synthetic raw materials. Thus, the present invention allows for an amount of 5 wt % or more of the alcohol component to be glycerolacetylester, which is predominantly monoacetylester and diacetylester.

In a preferred unsaturated polyester resin, the amount of glycerolacetylester is about 10 wt % or more, preferably about 20 wt % or more, and more preferably about 30 wt % or more of the alcohol component. It is possible to use the glycerol-acetylester mixture as (substantially) all of the alcohol component, although it may be preferred to use other aliphatic or aromatic diols.

In order to lower the tendency of the unsaturated polyester to show yellowing, it is preferred to have a wholly aliphatic unsaturated polyester. In contrast to the mono-acid benzoic acid, the glycerol-acetylester allows for the preparation of fully aliphatic unsaturated polyesters.

The glycerol and acetic acid mixture can be processed, for example with the organic tin based catalyst Fascat, at elevated temperature, like for example at 120° C. or higher, preferably 150° C. or higher, like for example 180° C. Generally, the temperature will be about 260° C. or lower, preferably about 220° C. or lower.

In a preferred embodiment, the invention relates to a process for making glycerol, mono-, di- and triacetylglycerolester in which the glycerol and acetic acid mixture is generally processed till an hydroxyl value of about 700 or lower is obtained, preferably about 600 or lower, and even more preferably about 550 or lower. Generally, the hydroxyl value will be about 300 or higher, preferably about 350 or higher, and most preferably about 400 or higher.

In a further preferred embodiment, the invention relates to a process for making glycerol, mono-, di- and triacetylglycerolester in which the glycerol and acetic acid mixture is generally processed till an acid value is obtained of about 60 or lower, preferably about 50 or lower, and most preferably about 40 or lower. Generally, the acid value will be about 5 or higher, like about 10 or higher.

During processing, it may be useful to correct for acetic acid that may evaporate, depending on the vessel and processing conditions.

The glycerol and acetic acid preferably are from natural sources. Glycerol can be obtained as side product from bio-diesel production. Acetic acid can be produced from fermentation of natural alcohol. Preferably, at least the glycerol is from a natural source.

Unsaturated polyesters can be prepared by condensation polymerization reaction techniques as are known in the art. Representative condensation polymerization reactions include polyesters prepared by the condensation of polyhydric alcohols and polycarboxylic acids or anhydrides. The polyalcohols part is also denoted as alcohol component; the polyacid part also as acid component. By adjusting the stoichiometry of the alcohols and the acids while maintaining an equivalent or excess of hydroxyl groups, hydroxy-functional polyesters can be readily produced to provide a wide range of desired molecular weights, unsaturation content and performance characteristics. In case the acid component is used in excess, an acid functional polyester is obtained.

The unsaturated polyester are derived from one or more aromatic and/or aliphatic polycarboxylic acids, the anhydrides thereof, and one or more aliphatic and/or aromatic polyols. The carboxylic acids include the saturated and unsaturated polycarboxylic acids and the derivatives thereof, such as maleic acid, fumaric acid, succinic acid, adipic acid, azelaic acid, dicyclopentadiene dicarboxylic acid, hexahydrophthalic anhydride, methylhexahydrophthalic anhydride, aromatic polycarboxylic acids, such as phthalic acid, isophthalic acid, terephthalic acid, etc. Anhydrides such as maleic anhydride, phthalic anhydride, trimellitic anhydride, or Nadic Methyl Anhydride (brand name for methylbicyclo[2.2.]heptene-2,3-dicarboxylic anhydride isomers) can also be used.

Representative saturated and unsaturated polyols which can be reacted with the carboxylic acids to produce hydroxy-functional polyesters include diols such as ethylene glycol, dipropylene glycol, 2,2,4-trimethyl 1,3-pentanediol, neopentyl glycol, 1,2-propanediol, 1,4-butanediol, 1,3-butanediol, 2,3-butanediol, 1,5-pentanediol, 1,6-hexanediol, 2,2-dimethyl-1,3-propanediol, 1,4-cyclohexanedimethanol, 1,2-cyclohexanedimethanol, 1,3-cyclohexanedimethanol, 1,4-bis(2-hydroxyethoxy)cyclohexane, trimethylene glycol, tetramethylene glycol, pentamethylene glycol, hexamethylene glycol, decamethylene glycol, diethylene glycol, triethylene glycol, tetraethylene glycol, norbornylene glycol, 1,4-benzenedimethanol, 1,4-benzenediethanol, 2,4-dimethyl-2-ethylenehexane-1,3-diol, 2-butene-1,4-diol, and polyols such as trimethylolethane, trimethylolpropane, trimethylolhexane, triethylolpropane, 1,2,4-butanetriol, glycerol, pentaerythritol, and dipentaerythritol.

At least part of the alcohol component is a acetylglycerolester mixture of the present invention.

Typically, the reaction between the polyols and the polycarboxylic acids is conducted at about 120° C. to about 250° C. in the presence or absence of an esterification catalyst such as dibutyl tin oxide.

Additionally, unsaturated polyesters can be prepared by substituting some or all of the polyols described above with epoxides and/or polyepoxides where acids and anhydride can open the oxirane ring to form the corresponding ester and hydroxy groups. Representative polyepoxides include ethyleneoxide, propyleneoxide and those prepared by condensing a polyhydric alcohol or polyhydric phenol with an epihalohydrin, such as epichlorohydrin, usually under alkaline conditions. Some of these condensation products are available commercially under the designations EPON or DER from Hexion Specialty Chemicals or Dow Chemical Company, respectively, and methods of preparation are representatively taught in U.S. Pat. Nos. 2,592,560; 2,582,985 and 2,694,694.

Another method to form unsaturated polyesters comprises chain extending the hydroxyl-functional polyesters by reacting the hydroxyl groups of a (precondensed) polyester with chain extenders, preferably polyalkylene oxide or lactones such as polyethylene oxide, polypropylene oxide or caprolactone, valerolactone, and butyrolactone.

Monocarboxylic acids can be used for the preparation of the unsaturated polyesters to control molecular weight, functionality, and other characteristic properties. The monocarboxylic acids can be aliphatic, cycloaliphatic, aromatic or mixtures thereof. Preferably, the monocarboxylic acid contains 6 to 18 carbon atoms, such as benzoic acid, hexahydrobenzoic acid, and mixtures thereof. The use of (additional) monocarboxylic acid may be in particular advantageous if a glycerolacetylester is used with a relatively high hydroxyl functionality.

Monohydroxy compounds can be used in the practice of this invention to control molecular weight, functionality, and other characteristic properties. Examples of suitable monofunctional alcohols include alcohols with 4-18 carbon atoms such as 2-ethyl butanol, pentanol, hexanol, dodecanol, cyclohexanol and trimethyl cyclohexanol.

Hydroxy-functional acids can be used to replace some and/or all of the acids and polyols described above. Typical hydroxy acids that can be used include dimethylol propionic acid and hydroxypivalic acid.

The unsaturated polyesters generally have an acid number of about 60 or lower, preferably of about 30 or lower. The unsaturated polyesters generally will have a hydroxyl value of about 100 or lower, preferably about 50 or lower.

The unsaturated polyesters generally will have a molecular weight of about 600 or higher, preferably about 1500 or higher. The molecular weight will be about 10000 or lower, preferably about 5000 or lower.

The unsaturated polyesters may be used in combination with vinylaromatic compounds and/or acrylic compounds. Preferred compounds are styrene, divinylbenzene, alpha-methylstyrene and the like. Styrene is most common and is most preferred. Examples of alkenically unsaturated monomers are styrene, substituted styrenes such as vinyl-toluene or tert.butylstyrene, (C₂-C₆)-alkylesters of acrylic acid and methacrylic acid, α-methylstyrene, cyclic acrylates and methacrylates, halogenated styrenes, 1-3-butanedioldimethacrylate and diallyl phthalate.

The unsaturation in the unsaturated polyester is preferably the polymerized residue of fumaric or maleic acid, and is a carbon-carbon double bond next to a carbonyl (C═O) group. Hence, polyesters that have only carbon-carbon unsaturations in fatty acids are not considered unsaturated polyesters in the present invention. In order to achieve sufficient unsaturation, it is preferred that about 10 mol % or more of the poly-acid component in the unsaturated polyester is a polymerized residue of fumaric or maleic acid, preferably about 40 mol % or more.

Substantial amount of the polyol component of the unsaturated polyester can be the glycerolacetate mixture of the present invention. It is preferred that at least 30 mol % of the polyolcomponent is the acetylglycerolester mixture obtainable per the present invention. It has been observed that the minor amount of triacetylglycerolester in the acetylester mixture may have a plasticizing effect. In case that effect is not aimed at, it is preferred to use such an amount of acetylester mixture, that less than about 10 wt % of triacetylester is present in the unsaturated polyester with styrene, preferably less than about 6 wt %, and more preferably less than 4 wt %.

The polyester resin generally is used with additives to form a compound that can be applied in or to a mold, which can be cured to form an article.

To enhance the physical properties, commonly glass fibres are used with the unsaturated polyester resin in the compound. Part or all of the glass fibre can optionally be replaced by carbon fibre, sisal, jute, asbestos, cotton, flax, hemp, organic synthetic fibres, such as polyamide, polyester, polypropylene or polyethylene, inorganic fibres such as quartz and beryllium and other metal fibres. The fibres may be present also in the form of continuous fibres or of a fibre mat, which is kept together by a suitable bonding agent, or in the form of. chopped filaments without binding agent. The length of the fibres used, particularly of the glass fibres,—if chopped fibres are used—may range from 0.5 mm to 50 mm. The fibre may be added in amounts of up to 80% (wt) (calculated on the total compound).

The compound with the resin may further comprise fillers. The fillers that can be used may be, for instance, marl, antimony trioxide, silica flour, coconut shell flour, talcum, calcium carbonate, silicon oxide, clay, calcium silicate, wood flour, glass beads, titanium dioxide, aluminium silicate, aluminium hydrate, carbon black or gypsum anhydrite. The filler content incorporated may range from 5 up to 90% by wt.

The resin can be used together with a catalyst for curing the resin to an article. The catalyst applied may comprise, for instance, tert-butylperbenzoate, benzoyl peroxide, tert-butylperoxide, tert-butylperoctoate, di-tertbutylperoctoate, cyclohexanone peroxide, methylethylketone peroxide, acetylacetone peroxide or lauroylperoxide, combinations of these, optionally with hydrogen peroxide. Other suitable catalysts are UV sensitive initiators.

Further additives may comprise inhibitors, accelerators, release agents and low profile agents. Inhibitors are often used to provide sufficient stability of the moulding compound at ambient, temperature before the moulding process, the inhibitors also leave enough time for the flowing into the mould before the gelling commences. Examples of such inhibitors are hydroquinone and p-benzoquinone. Examples of accelerators are octoates, naphthenates and amines, such as cobaltoctoate, dimethylaniline, diethylaniline and dimethyl para-toluidine. Suitable release agents are known, such as the stearates of zinc, calcium or aluminium, phosphates, silicons, polyvinylalcohol and waxes. Semi-permanent release agents can be used as well. Usual low-profile additives are, for instance, thermoplastics. Examples of thermoplastics are homopolymers of methyl-methacrylate, ethylmethacrylate and butylmethacrylate, methylacrylate and ethylacrylate, styrene, copolymers of methylmethacrylate and other low-molecular weight alkylacrylates and alkyl20 methacrylates and copolymers of methylmethacrylate with small amounts of one or more of the following monomers: laurylmethacrylate, isobornylmethacrylate, acrylamide, hydroxyethyl-methacrylate, styrene, 2-ethylhexylacrylate, acrylonitrile, methacrylic acid, methacrylamide, methylolacrylamide and cetylstearylmethacrylate, or copolymers of styrene and acrylonitrile, copolymers of vinylchloride and vinylacetate, cellulose acetate butyrate, cellulose acetate proprionate and styrene maleic anhydride copolymer.

Further, the usual pigments or colourants can be added.

The invention is exemplified in the following examples, without being limited thereto.

### EXAMPLES

### Examples 1-3

Acetic acid (HAc) and glycerol (Gly) are charged into a reaction flask, together with the catalyst, and are reacted at 120-130° C. for one hour, until distillate is no longer recovered. Thereafter, the temperature is increased stepwise up to 170-180° C. while keeping the still head temperature at 96-102° C. The distillate is at intervals, titrated to determine the acid content, and lost acetic acid is charged to the reaction vessel in examples 1 and 2. In example 3, no acetic acid is charged back. Processing is continued till the distillate reaches the theoretical value. The mole ratio of acid and glycerol charged, and the amounts of glycerol, mono-, di- and triacetylester are given in table 1, for three batches.

TABLE 1

| | Mole | | | | | | |
|---|---|---|---|---|---|---|---|
| | ratio | | OH | | | | |
| Example | HAc:Gly | AV | number | Gly | Mono | Di | Tri |
| 1 | 1.75:1 | 45 | 523 | 12.04 | 34.0 | 43.36 | 10.59 |
| 2 | 2:1 | 23.5 | 430 | 6.20 | 32.82 | 47.30 | 13.68 |
| 3 | 2:1 | 57 | 560 | 16.20 | 38.83 | 38.07 | 6.90 |

### Example 4-10 and Comparative Example A

With the glycerol-acetylester mixtures from examples 1 and 2, unsaturated polyesters were prepared, while using the catalyst form the glycerolacetylester synthesis. The components are as given in table 2.

TABLE 2

| Reagent | Example | | | | | | | |
|---|---|---|---|---|---|---|---|---|
| (mol) | A | 4 | 5*** | 6 | 7 | 8 | 9 | 10 |
| Glycerol- | 7.07 | | | | | | | |
| BA* | | | | | | | | |
| Glycol | | 3.89 | 32.00 | 8.40 | 5.45 | 10.61 | | |
| example 1** | | | | | | | | |
| Glycol | | | | | | | 22.92 | 27.78 |
| example 2** | | | | | | | | |
| Maleic | 10.20 | 6.08 | 50.00 | | 2.72 | | 11.46 | |
| anhydride | | | | | | | | |
| Fumaric | | | | 12.01 | | 5.30 | | 13.79 |
| acid | | | | | | | | |
| Propylene | 6.58 | 3.62 | 34.6 | 7.84 | | | | |
| glycol | | | | | | | | |
| Styrene | 16.46 | 4.83 | 61.05 | 13.46 | 2.57 | 5.73 | 14.19 | 17.20 |

*glycerol BA is glycerol reacted with two moles of benzoic acid (BA)
**glycols were charged on the basis of equivalent molecular weights (that is molecular weight per OH functionality, calculated from measured OH numbers).
***Resins of examples 4 and 5 are the same formulation processed to different end points.

All the resin formulations contained the same levels of additives, namely:
Catalyst Fascat 4102 at a concentration of 0.13 wt % on BA and 0.3 wt % on HAc.
THQ 33% solution at 100 ppm on total weight
Triphenyl phosphate at 100 ppm on total weight
Copper naphthenate at 33 ppm on total weight
Sodium acetate (etherification inhibitor) at 50 ppm on base resin's weight

For resin A, the reaction vessel was heated till 160° C. and held at this temperature for 1 hour. Thereafter, the temperature was gradually increased to 220° C. After one hour, while distilling, xylene was added as azeotropic agent. Processing was continued until the amount of recovered water was about 80%. The reaction mixture is gradually cooled. The resins 4-10 were processed as described in Example 1. The final acid value was about 60 or less. The unsaturated polyester was blended with styrene at about 60° C., further cooled, and stored in a steel container.

The resins have the properties, as shown in Table 3

| Resin | A | 4 | 5 | 6 | 7 | 8 | 9 | 10 |
|---|---|---|---|---|---|---|---|---|
| Non-styrenated | | | | | | | | |
| resin | | | | | | | | |
| Acid value | 28 | 40 | 28 | 31 | 31 | 29 | 22 | 26 |
| (mg/g KOH) | | | | | | | | |
| ICI viscosity at | 36 | 3 | 22 | 19 | 1.5 | 3 | 20 | 15 |
| 75° C. (Poise) | | | | | | | | |
| Styrenated | | | | | | | | |
| resin | | | | | | | | |
| Appearance of | Clear | Clear | Clear | Clear | Hazy | Clear | Clear | Clear |
| liquid Resin | yellow | yellow | yellow | yellow | yellow | yellow | yellow | yellow |
| ICI viscosity | 3.4 | 4.5 | 4.5 | 4.8 | 3.2 | 4.6 | 4.6 | 4.6 |
| (Poise) | | | | | | | | |
| Gel time at | 5.67 | 5.6 | 5.0 | 4.4 | 5.5 | 4.9 | 4.0 | 3.7 |
| 25° C. (min)* | | | | | | | | |
| Exotherm | 14.4 | 17.4 | 15.5 | 15.3 | 13.7 | 12.0 | 16.2 | 13.8 |
| time (min)* | | | | | | | | |
| Exotherm | 117.5 | 142 | 148 | 140 | 115 | 118 | 118 | 132 |
| temperature (° C.)* | | | | | | | | |
| Styrene | 32.0 | 26.0 | 37.0 | 34.4 | 21.6 | 24.5 | 30.5 | 30.0 |
| content (wt %)** | | | | | | | | |
| Liquid density, | 1.113 | 1.126 | 1.095 | 1.106 | 1.161 | 1.155 | 1.134 | 1.138 |
| 20° C. (g/ml) | | | | | | | | |
| Solid density, | 1.222 | 1.244 | 1.200 | 1.209 | 1.257 | 1.256 | 1.250 | 1.256 |
| 20° C. (g/ml) | | | | | | | | |
| Volumetric | 8.92 | 8.15 | 8.59 | 8.56 | 7.59 | 8.05 | 9.29 | 9.40 |
| shrinkage | | | | | | | | |

When cured, the unsaturated polyester had the following properties DMTA results are given in Table 4

| | | | | v_{c} × 10²⁵ |
|---|---|---|---|---|
| Resin | T_{α} | E′ᵣ (MPa) | M_{c} (g/mol) | (chains/m³) |
| A | 101 | 10.0 | 1293.0 | 56.9 |
| 4 | 126 | 60.8 | 229.6 | 326.3 |
| 6 | 150 | 63.8 | 223.6 | 325.6 |
| 7 | 25, 58 | 9.6 | 1135.4 | 66.7 |
| 8 | 80 | 28.8 | 439.1 | 172.2 |
| 9 | 100 | 32.8 | 403.3 | 186.7 |
| 10 | 117 | 38.2 | 361.6 | 209.2 |
| M | | | | |

T_{α} = glass transition temperature
E′ᵣ = the elastic storage modulus in the plateau region
M_{c} = number-average molecular weight between cross-linked junctions
v_{c} = cross-link density

Heat deflection temperature is given in table 5

| Resin | HDT (° C.) |
|---|---|
| A | 56.5 |
| 4 | 73.5 |
| 5 | 88.5 |
| 6 | 87.0 |
| 8 | 37.5 |
| 9 | 54.0 |
| 10 | 62.0 |

Resin 7's HDT was too low to be measured with available equipment, as the material already deflected under the load at room temperature (even at ˜10° C.).
Tensile properties are given in Table 6

| | Tensile strength | Tensile modulus | Strain at |
|---|---|---|---|
| Resin | (MPa) | (GPa) | break (%) |
| A | 42.0 ± 3.4 | 2.9 ± 0.2 | 2.2 ± 0.4 |
| 4 | 22.3 ± 2.1 | 2.5 ± 0.1 | 1.0 ± 0.1 |
| 5 | 28.4 ± 1.4 | 2.6 ± 0.1 | 1.2 ± 0.1 |
| 6 | 27.3 ± 2.9 | 2.9 ± 0.1 | 1.0 ± 0.2 |
| 7 | 4.2 ± 0.3 | 1.5 ± 0.1 | 5.8 ± 0.5 |
| 8 | 13.6 ± 0.6 | 0.9 ± 0.0 | 2.2 ± 0.3 |
| 9 | 25.3 ± 3.1 | 1.8 ± 0.1 | 2.3 ± 0.5 |
| 10 | 22.9 ± 2.8 | 2.0 ± 0.3 | 1.4 ± 0.3 |

### Examples 11-16 and Comparative Example B

The resins were used to make glass fibre reinforced laminates. Laminates were prepared from chopped strand mat glass fibre and resin catalysed with 0.15% cobalt octoate (6% solution) and 1% MEKP, using conventional hand lay-up technique. Laminates were cured at room temperature, followed by post-curing at 85° C. for 2 hours. Properties are given in Table 7.

| | | Tensile strength | Tensile modulus | |
|---|---|---|---|---|
| Resin | Example | (MPa) | (GPa) | Strain* (%) |
| A | B | 88.5 ± 16.7 | 6.8 ± 1.0 | 1.9 ± 0.1 |
| 4 | 11 | 74.8 ± 7.8 | 6.6 ± 0.5 | 1.8 ± 0.2 |
| 6 | 12 | 58.6 ± 5.0 | 5.7 ± 0.4 | 1.8 ± 0.2 |
| 7 | 13 | 54.3 ± 9.3 | 3.6 ± 0.6 | 1.8 ± 0.3 |
| 8 | 14 | 71.6 ± 5.0 | 4.3 ± 0.2 | 2.0 ± 0.2 |
| 9 | 15 | 73.6 ± 7.6 | 5.1 ± 0.5 | 1.7 ± 0.3 |
| 10 | 16 | 78.2 ± 7.5 | 5.5 ± 0.5 | 2.0 ± 0.1 |

* The strain was measured at Max Tensile strength and not at complete severing of test specimens.

The above examples show that glycerolacetates (acetins) can be used as alcohol component in unsaturated polyester manufacture while keeping good properties.

1. A process for the preparation of glycerolacetylesters, in which acetic acid and glycerol having a molar ratio of less than 2.5, are reacted in the presence of a stannous catalyst to produce a mixture of glycerol, mono-, di- and triacetylglycerolester.

2. The process according to claim 1, wherein the resulting amount of triester relative to the resulting amounts of glycerol, monoester and diester is less than 15 mol %.

3. The process according to claim 1, wherein the resulting amount of glycerol relative to the resulting amounts of, monoester, diester and triester is less than 25 mol %.

4. The process according to claim 1, wherein the resulting amount of mono-ester relative to the resulting amounts of glycerol, diester and triester is about 20 mol % or more and wherein the resulting amount of mono-ester relative to the resulting amounts of glycerol, diester and triester is about 50 wt % or less.

5. The process according to claim 1, wherein the resulting amount of di-ester relative to the resulting amounts of glycerol, monoester and triester is about 20 mol % or more, and wherein the resulting amount of di-ester relative to the resulting amounts of glycerol, monoester and triester is about 60 wt % or less.

6. The process according to claim 1, wherein the resulting mixture has a hydroxyl value of about 700 or lower, and wherein the hydroxyl value is about 300 or higher.

7. The process according to claim 1, wherein the resulting mixture has an acid value of about 60 or lower, and wherein the acid value is about 5 or higher.

8. The process according to claim 1, wherein the resulting amount of triester relative to the resulting amounts of glycerol, monoester and diester is greater than 1.0 mol %, and wherein the resulting amount of glycerol relative to the resulting amounts of, monoester, diester and triester is about 5 mol % or more.

9. (canceled)

10. The process according to claim 1, wherein the mixture of glycerol, mono-, di- and triacetylglycerolester is polymerizable through radical polymerization to form articles in the preparation of unsaturated polyesters.

11. An unsaturated polyester comprising more than 5 wt % of the glycerolacetylester obtainable by the process according to claim 1 copolymerized in a polymer.

12. An unsaturated polyester according to claim 11, wherein the unsaturated polyester contains more than 10 mol % of the acid component the polymerized residue of maleic or fumaric acid.

13. An unsaturated polyester according to claim 11, wherein the unsaturated polyester contains styrene or another reactive diluent.

14. An unsaturated polyester according to claim 1, wherein the unsaturated polyester further contains at least one of: fibres, fillers and catalysts.

15. A process of making an unsaturated polyester by reacting an α,β-unsaturated carboxylic acid and at least a glycerolacetylester, wherein mono- and diacetylesters of glycerol are made from glycerol and acetic acid using an organotin catalyst, and wherein in making the unsaturated polyester the same organotin catalyst is used.

16. A mixture of glycerol, mono-, di- and triacetylglycerolesters prepared by reacting acetic acid and glycerol having a molar ratio of less than 2.5 in the presence of a stannous catalyst.

17. An unsaturated polyester comprising more than 5 wt % of the mixture of the glycerol, mono-, di- and triacetylglycerolesters of claim 16 copolymerized in a polymer.

18. The mixture according to claim 16, wherein the amount of triester relative to the amounts of glycerol, monoester and diester is less than 15 mol %.

19. The mixture according to claim 16, wherein the amount of glycerol relative to the amounts of monoester, diester and triester is less than 25 mol %.

20. The mixture according to claim 16, wherein the amount of mono-ester relative to the amounts of glycerol, diester and triester is about 20 mol % or more and wherein the amount of mono-ester relative to the amounts of glycerol, diester and triester is about 50 wt % or less.

21. The mixture according to claim 16, wherein the amount of di-ester relative to the amounts of glycerol, monoester and triester is about 20 mol % or more, and wherein the amount of di-ester relative to the amounts of glycerol, monoester and triester is about 60 wt % or less.

22. The process according to claim 16, wherein the mixture has a hydroxyl value of about 700 or lower, and wherein the hydroxyl value is about 300 or higher.

23. The mixture according to claim 16, wherein the mixture has an acid value of about 60 or lower, and wherein the acid value is about 5 or higher.

24. The mixture according to claim 16, wherein the amount of triester relative to the amounts of glycerol, monoester and diester is greater than 1.0 mol %, and wherein the amount of glycerol relative to the amounts of, monoester, diester and triester is about 5 mol % or more.
